(19) United States
(12) Patent Application Publication
(10) Pub. No.: US 2024/0366070 A1
(43) Pub. Date: Nov. 7, 2024

(54) OROPHARYNGEAL CANNULA COMPATIBLE WITH BITE GUARD

(71) Applicant: INTERMED LABS LLC, Morgantown, WV (US)

(72) Inventors: M'hamed TURKI, Huntington, WV (US); Justin R. CHAMBERS, Morgantown, WV (US); William T. McCLELLAN, Morgantown, WV (US)

(21) Appl. No.: 18/654,119

(22) Filed: May 3, 2024

Related U.S. Application Data
(60) Provisional application No. 63/464,296, filed on May 5, 2023.

(51) Int. Cl. A61B 1/00; A61B 1/273; A61M 25/06

(52) U.S. Cl. CPC A61B 1/00154; A61B 1/2736; A61M 25/0662

(57) ABSTRACT

A oropharyngeal cannula device configured to be inserted into a bite guard includes: a first portion configured to engage the bite guard while positioned in a channel extending through the bite guard, wherein the first portion defines a passageway inside the channel; and a second portion extending from the first portion and configured to directly contact a superior surface of a tongue of a patient, while the bite guard is in a mouth of the patient, to prevent collapse of an oropharyngeal airway of the patient.

### PRIORITY

This application claims priority to U.S. provisional patent application No. 63/464,296 filed May 5, 2023, the contents of which are incorporated by reference herein in their entirety.

### FIELD OF THE INVENTION

Aspects of the present invention relate to an oropharyngeal cannula, and more particularly to an oropharyngeal cannula compatible with a bite guard (also called a bite block).

### BACKGROUND

A gastroscopy procedure, also known as an upper endoscopy or esophagogastroduodenoscopy (EGD), is a diagnostic and therapeutic tool used in gastroenterology to examine the upper part of the gastrointestinal (GI) tract, which includes the esophagus, stomach, and duodenum. This procedure is crucial for diagnosing and managing various disorders of the upper GI tract. When performing these and other endoluminal exploration procedures through the mouth (e.g., an upper endoscopy, transesophageal echocardiogram, bronchoscopy, etc.), it is common practice to use a device such as a bite guard that protects the patient's teeth from trauma by the endoscope and that protects the endoscope from damage from biting.

Management of the airway during a gastroscopy procedure is a critical aspect, especially when sedation is involved. Patients with a history of sleep apnea can be especially vulnerable to losing the airway during sedation. For this reason, when anesthesia is performed on patients with obstructive sleep apnea, an oropharyngeal cannula, a.k.a. Guedel cannula, is usually placed in the patient's mouth to prevent hypoxia events. However, when performing an endoluminal exploration procedure through the mouth under anesthesia and with a bite guard in place, using an oropharyngeal cannula is not possible due to the limited space within the bite guard channel, this space being occupied by the endoscope. Therefore, it would be desirable to have a device that can be inserted into a bite guard, maintain the airway, and be able to pass the endoscope.

### SUMMARY

Implementations of the invention address the above-noted problems of the prior art by providing an oropharyngeal cannula that can be placed through a bite guard while still allowing endoscopic exploration using an endoscope passing through the same bite guard concurrently with the oropharyngeal cannula.

According to an aspect of the present invention, there is provided an oropharyngeal cannula to help prevent the collapse of the oropharyngeal airways during an endoscopic examination that can be placed on a bite guard through a system of foldable wings. In some embodiments, the oropharyngeal cannula includes one or more oxygen channels, extending through the length of the device, allowing the delivery of oxygen directly into the oropharyngeal space. The oropharyngeal cannula may come in different sizes.

Thus, the oropharyngeal cannula can be placed through the bite guard after sedating the patient and can be connected to the oxygen source in its version with oxygen tunnels.

According to an aspect of the invention, there is an oropharyngeal cannula device configured to be inserted into a bite guard, the device comprising: a first portion configured to engage the bite guard while positioned in a channel extending through the bite guard, wherein the first portion defines a passageway inside the channel; and a second portion extending from the first portion and configured to directly contact a superior surface of a tongue of a patient, while the bite guard is in a mouth of the patient, to prevent collapse of an oropharyngeal airway of the patient. In embodiments, the passageway is sized to accommodate passing an endoscope through the passageway.

In an embodiment, the first portion comprises: a body; a first wing extending from a first lateral side of the body; and a second wing extending from a second lateral side of the body, and the second portion comprises a tongue holder extending from a backside of the body. In embodiments, the first wing and the second wing are foldable between a first configuration in which the first wing and the second wing extend laterally outward from the body and a second configuration in which the first wing and the second wing are curved to a shape corresponding to an interior surface of the channel of the bite guard. In embodiments, the first wing and the second wing each include one or more protrusions configured to engage an internal wall of the bite guard. In embodiments, the device further comprises a flange extending from a front side of the body. In embodiments, the device further comprises one or more air channels extending continuously through the body and the tongue holder.

In an embodiment, the first portion comprises a front adaptor, and the second portion comprises a guide. In an embodiment, the guide comprises an upper guide and a lower guide that define a guide passageway between the upper guide and the lower guide. In embodiments, the at least one of the upper guide and the lower guide is resiliently affixed to the front adaptor, and the at least one of the upper guide and the lower guide is configured to flex relative to the front adaptor in response to an endoscope having an outer diameter greater than a dimension of the guide passageway passing through the guide passageway. In embodiments, the front adaptor is configured to engage the bite guard via friction fit. In embodiments, the front adaptor comprises an outer surface having a shape that corresponds to a shape of an interior surface of the channel of the bite guard and that directly contacts the interior surface of the channel of the bite guard when the front adaptor engages the bite guard. In embodiments, the front adaptor has a size and shape that prevents rotation of the device relative to the bite guard when the front adaptor is engaged with the bite guard, and the guide has a size and shape that permits rotation of the device relative to the bite guard when the guide is inside the channel of the bite guard and the front adaptor is not engaged with the bite guard. In embodiments, the guide comprises: a lower surface configured to directly contact the superior surface of the tongue of the patient when the first portion is engaged with the bite guard in the mouth of the patient; and an upper surface opposite the lower surface. In an embodiment, the lower surface and the upper surface are flat. In an embodiment, the lower surface is convex and the upper surface is concave. In an embodiment, a first portion of the lower surface is convex and a second portion of the lower surface is flat, and a first portion of the upper surface is concave and a second portion of the upper surface is flat. In an embodiment, the device further comprises a flange extending outward from the front adaptor on a side of the front adaptor opposite the guide.

In accordance with aspects of the invention, a method of using the oropharyngeal cannula device includes: inserting the oropharyngeal cannula device into the bite guard that is in the mouth of the patient, wherein the inserting comprises advancing the second portion of the oropharyngeal cannula device through the channel of the bite guard and causing the first portion of the oropharyngeal cannula device to engage the bite guard. In embodiments, the method further includes passing an endoscope through the passageway of the oropharyngeal cannula device while the oropharyngeal cannula device is inside the channel of the bite guard.

### BRIEF DESCRIPTION OF THE SEVERAL VIEWS OF THE DRAWINGS

Aspects of the present invention are described in the detailed description which follows, in reference to the noted plurality of drawings by way of non-limiting examples of exemplary embodiments of the present invention.

FIG. 1 illustrates a top and posterior perspective view of an oropharyngeal cannula in its open configuration according to an embodiment of the invention.

FIG. 2 illustrates a top and front perspective view of an oropharyngeal cannula in its open configuration according to an embodiment of the invention.

FIG. 3 illustrates a bottom and front perspective view of an oropharyngeal cannula in its open configuration according to an embodiment of the invention.

FIG. 4 illustrates a front view of an oropharyngeal cannula in its open configuration according to an embodiment of the invention.

FIG. 5 illustrates a magnified front view of a wing with emphasis on the sweep hooks according to an embodiment of the invention.

FIG. 6 illustrates a top and posterior perspective view of an oropharyngeal cannula in its folded configuration according to an embodiment of the invention.

FIG. 7 illustrates a front view of an oropharyngeal cannula in its folded configuration according to an embodiment of the invention.

FIG. 8 illustrates a top and posterior view of an oropharyngeal cannula inserted in a bite guard according to an embodiment of the invention.

FIG. 9 illustrates a side view of an oropharyngeal cannula inserted in a bite guard according to an embodiment of the invention.

FIG. 10 illustrates a posterior view of an oropharyngeal cannula inserted in a bite guard according to an embodiment of the invention.

FIG. 11 illustrates a top and front perspective view of an oropharyngeal cannula with two oxygen channels in its open configuration according to an embodiment of the invention.

FIG. 12 illustrates a top and posterior perspective view of an oropharyngeal cannula with two oxygen channels in its open configuration according to an embodiment of the invention.

FIG. 13 illustrates a bottom perspective view of an oropharyngeal cannula with two oxygen channels in its open configuration with a transverse cut in the midsection of the tongue holder according to an embodiment of the invention.

FIG. 14 illustrates a top view of the oropharyngeal cannula with its defining orientation and central axis according to an embodiment of the invention.

FIGS. 15-19 illustrate an oropharyngeal cannula configured to be used with a bite guard according to an embodiment of the invention.

FIGS. 20-22 illustrate variations of the oropharyngeal cannula according to embodiments of the invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details in more detail than is necessary for the fundamental understanding of aspects of the present invention, the description taken with the drawings making apparent to those skilled in the art how several forms of the present invention may be embodied in practice.

This description provides an oropharyngeal cannula that can be placed through a bite guard. Through-the-mouth endoscopic explorations remain possible while the cannula is in place in the bite guard thanks to its hollow structure. A system of foldable wings allows the oropharyngeal cannula to be inserted through the opening of the bite guard and will limit the vertical movements of the oropharyngeal cannula. A rim of sweep hooks at the distal and external side of the wings anchors the device to the bite guard to oppose disinsertion forces.

In some embodiments, one or two oxygen channels have been added to the device. The channels travels longitudinally through the device with a proximal and a distal opening. An oxygen source can be connected to the proximal opening and would deliver the oxygen directly into the oropharyngeal space.

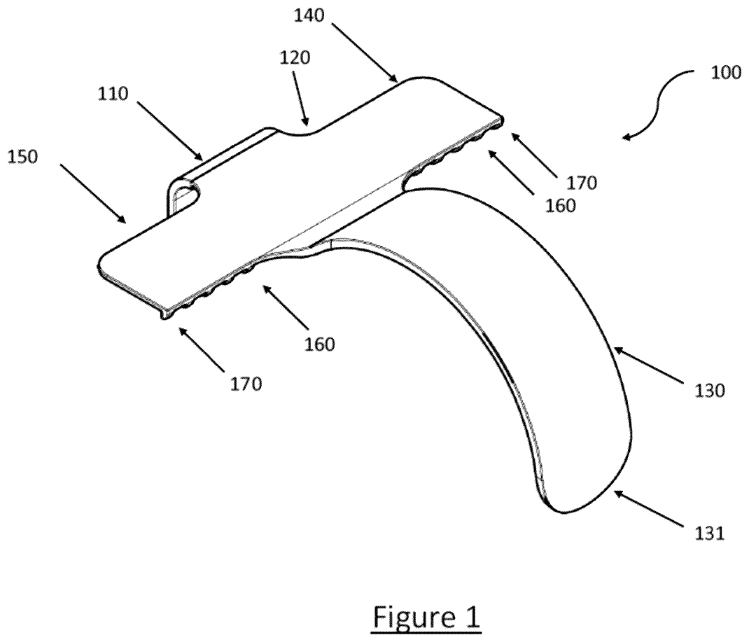

FIG. 1 shows an oropharyngeal cannula in its open configuration indicated generally at 100. The oropharyngeal cannula 100 includes a retention flange 110, a body 120, a tongue holder 130 and two wings 140 & 150 with sweep hooks 160 & 170 extending from the inferior walls of the wings 140 & 150. In some examples, the cannula comprises a single solid form. The cannula 100 may comprise one or more suitable materials such as a polymer, rubber, silicon and the like. In specific examples, the cannula 100 is manufactured using injection molding. The retention flange 110 leans against the external wall of the bite guard to keep the cannula 100 at the level of the lips. The body 120 is the central portion of the cannula 100 connecting the retention flange 110 to the tongue holder 130 longitudinally and the wings 140 & 150 laterally. The tongue holder 130 retains the tongue anteriorly and inferiorly. The wings 140 & 150 lean on the inner walls of the bite guard opening. The sweep hooks 160 & 170 oppose the oropharyngeal cannula disinsertion by providing a rim resting against the internal wall of the bite guard.

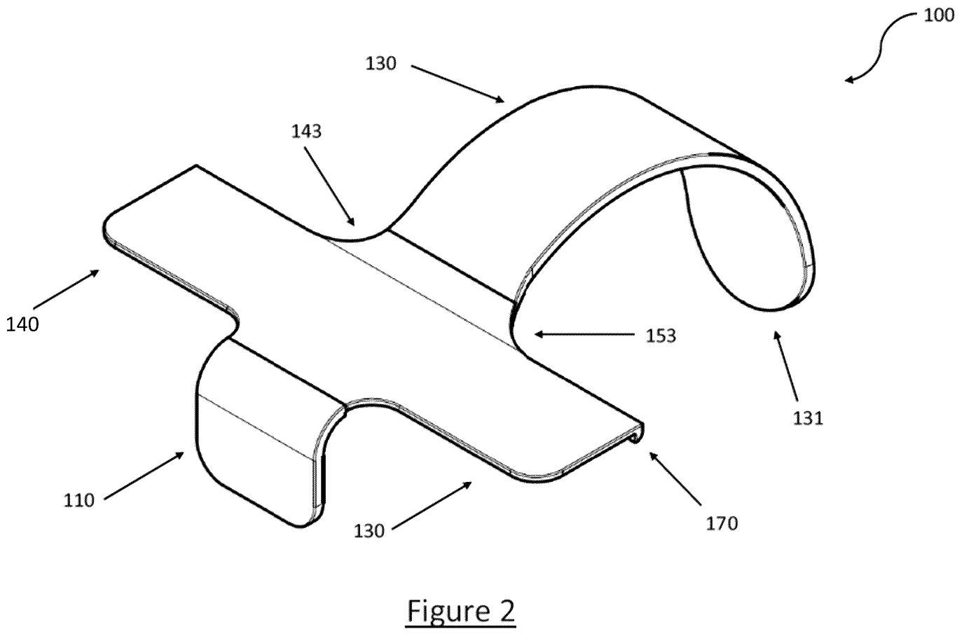

Turning to FIG. 2, the retention flange 110 is now described in greater detail. In general, the flange 110 provides an external barrier preventing the cannula 100 from sliding beyond the oropharyngeal area. When resting against the external wall of the bite guard (shown later in FIG. 8), the flange 110 will help generate the opposing force holding the tongue forwards. In the present embodiment, the flange 110 has a rectangular shape with smooth edges, however the flange is not particularly limited. The flange 110 may extend from the body 120 at approximately 90°. The angle between the extension flange 110 and the body 120 is smooth in this representation. The flange 110 can take on a variety of shapes, provided the shape offers a resting surface against the external wall of the bite guard. In the present embodiment, the flange 110 is made of a rigid plastic material. However, other materials for the flange 110 are contemplated, such as silicon and rubber, provided that these are sufficiently resistant to keep the cannula 100 from sliding forward.

The tongue holder 130 is now described in greater detail. In general, the tongue holder 130 may be substantially planar with a concave aspect toward the bottom. The tongue holder 130 is in direct contact with the superior surface of the tongue. The tongue holder 130 exerts downward and forward pressures on the tongue. This downward pressure widens the view during oral intubation. Both of the forward and downward pressures help prevent the tongue from occluding the oropharyngeal area. The tongue holder 130 may be a longitudinal extension of the body 120. In the present embodiment, the tongue holder 130 is a thin blade of hard plastic with a smooth surface. Also in the present embodiment, the tongue holder 130 ends in a rounded tip 131 to avoid mucosal trauma during insertion. The tongue holder 130 can take on a variety of shapes, provided the shape offers a downward and forward pressure on the tongue with no to minimal trauma. Preferably, the tongue holder 130 is made of the same material as the body 120 and the flange 110. However, other materials are contemplated.

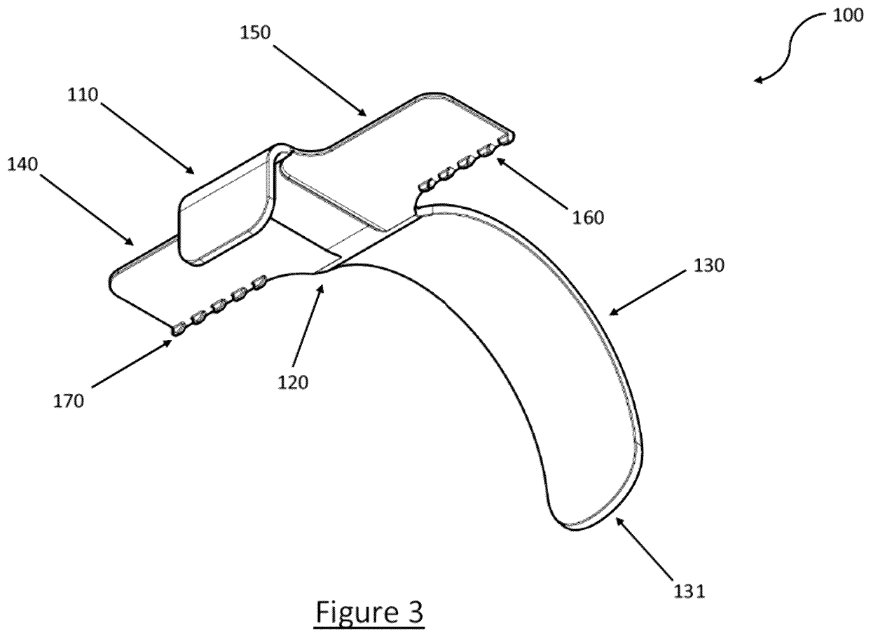

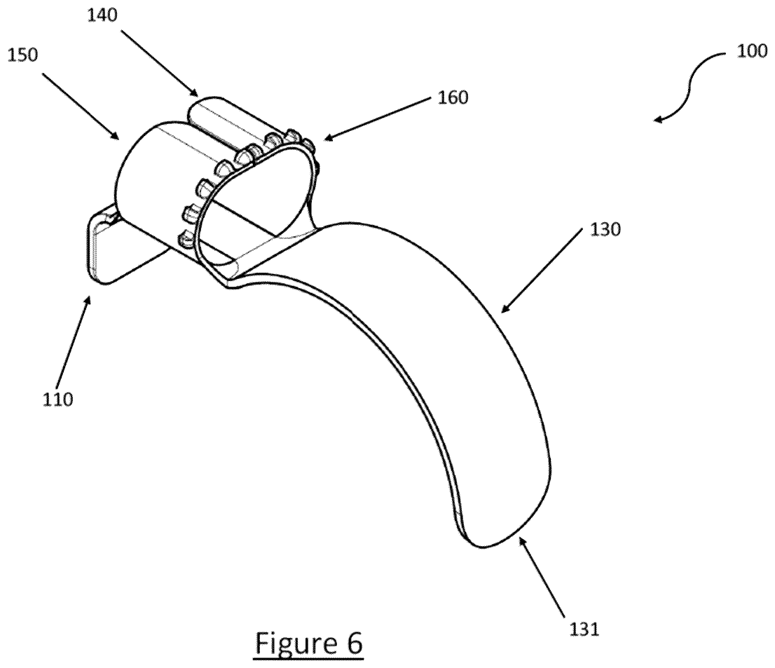

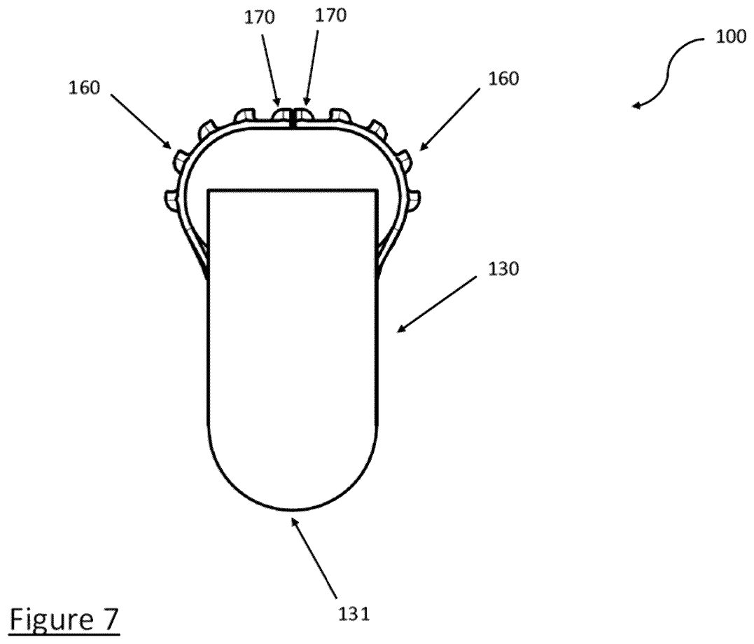

The wings are now described in greater detail. The oropharyngeal cannula has two flexible wings 140 & 150 both extending from the body 120. Before insertion into the bite guard, the wings 140 & 150 may be flat, as shown in FIG. 2. In this embodiment, the wings 140 & 150 are substantially rectangular in shape, however the shape of wings 140, 150 is not particularly limited. The wings 140 & 150 may include a line of sweep hooks 160 & 170 on the inferior surface of each wing 140 & 150, as shown in FIG. 3. The wings 140 & 150 are not perfectly rectangular; their internal edges 143 & 153 are slightly incurvated at their connection with the body. When the oropharyngeal cannula 100 is inserted into a bite guard, the wings 140 & 150 are first folded inward to pass through the canal of the bite guard, as shown in FIGS. 6 and 7. Once inserted, the wings 140 & 150 are configured to contact the internal wall of the bite guard. Through this pipe-in-pipe system, the folded wings 140 & 150 will help stabilize the body 120 against the inferior wall of the bite guard by opposing vertical ascending pressure applied by the tongue. The length of the wings 140 & 150 and the body 120 can be slightly longer than the length of the bite guard canal; this would offer easier maneuverability in case the cannula 100 has to be removed without removing the bite guard. The wings 140 & 150 are made of foldable plastic material in this representation, however, other materials for the wings 140 & 150 are contemplated, such as silicon and rubber.

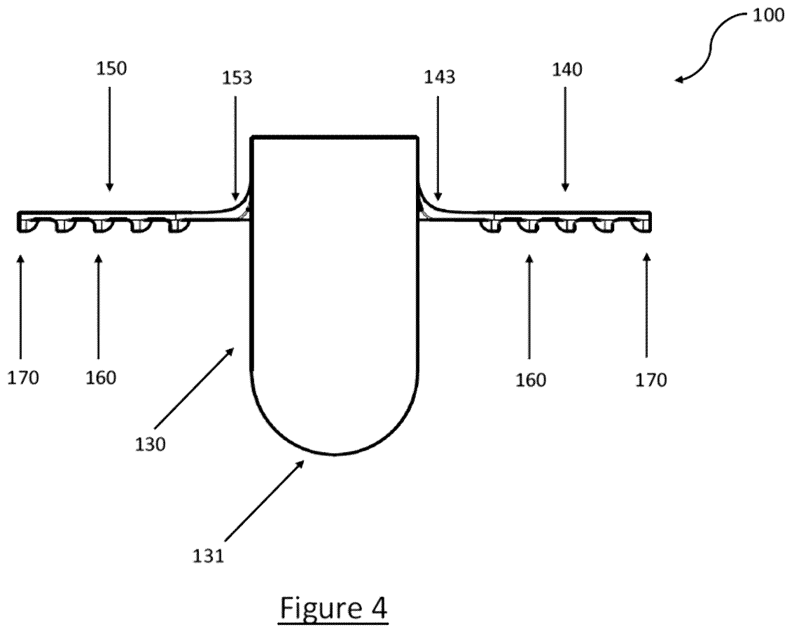

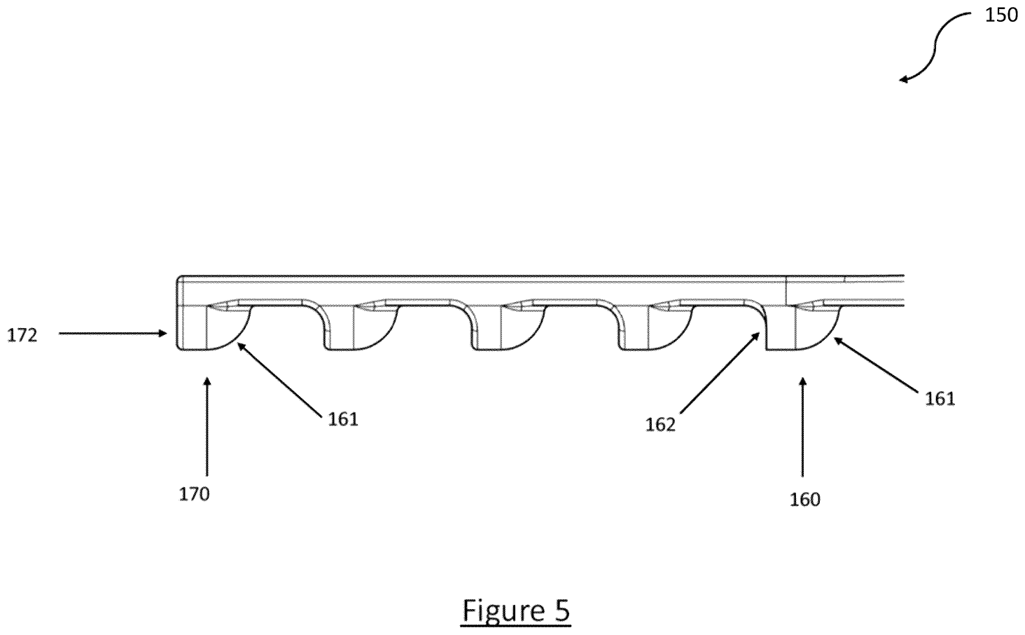

The sweep hooks 160 & 170 are described in greater detail. There are essentially two types of sweep hooks in the embodiment shown in FIGS. 1-6: standard sweep hooks 160 and terminal sweep hooks 170. In this representation, each wing 140 & 150 includes four standard sweep hooks 160 and one terminal sweep hook 170. Based on representations, the number of standard sweep hooks can vary based on the size of the wings. Both types of sweep hooks are half domes with their convexity 161 oriented toward the central axis A-A of the cannula 100. This smooth curve is designed to help with sliding during insertion. After insertion and wings expansion, the sweep hooks 160 & 170 will apply pressure against the internal wall of the bite guard to help oppose disinsertion forces. The terminal sweep hooks 170 differ from the standard sweep hooks 160 at their base; the terminal sweep hooks 170 join to the wings 140 & 150 at substantially right angles on their flat side 172 while the flat sides 162 of standard sweep hooks 160 include a curved portion. The design of the terminal sweep hooks 170 allows a coaptation of the terminal sweep hooks 170 after insertion. This coaptation may or may not take place depending on the type of bite guard used and its diameter.

The body 120 is now described in greater detail. In general, the body 120 represents a central piece in the oropharyngeal cannula. The body 120 may be connected externally to the flange 110 and internally to the tongue holder 130. The body 120 is a continuum between these two entities. Laterally, the body 120 has both wings 140 & 150 attached to it. Although the body 120 is exemplified by a flat rectangular surface, the body 120 can take a variety of shapes, provided the shape is complementary to the flange 110, the tongue holder 130 and the wings 140 & 150. In the present embodiment, the body 120 is made of plastic material. However, other materials for the body 120 are contemplated, such as silicon and rubber, provided that the body 120 is compatible with the other portions of the cannula 100.

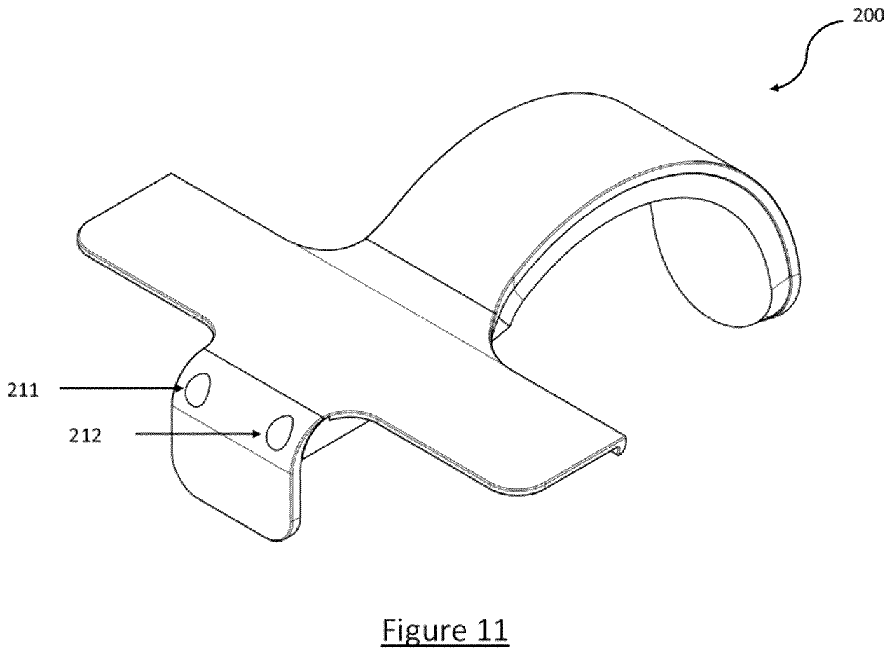

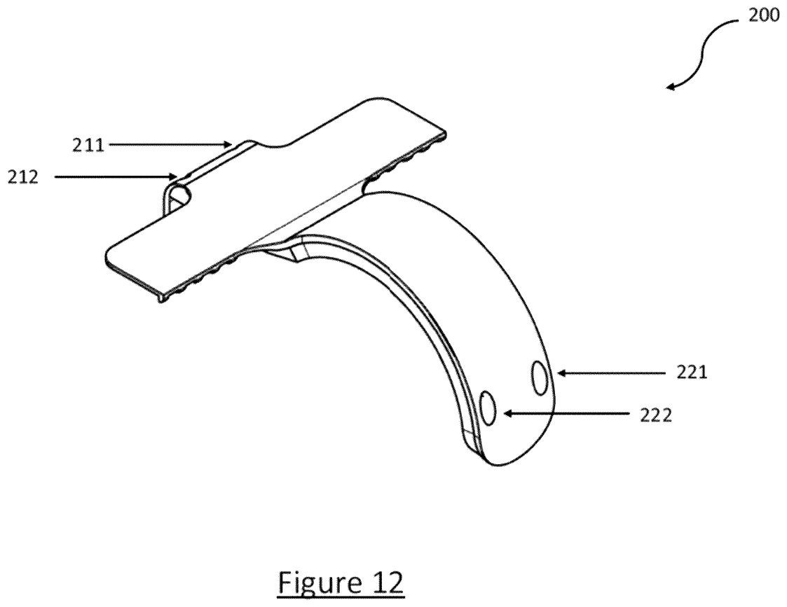

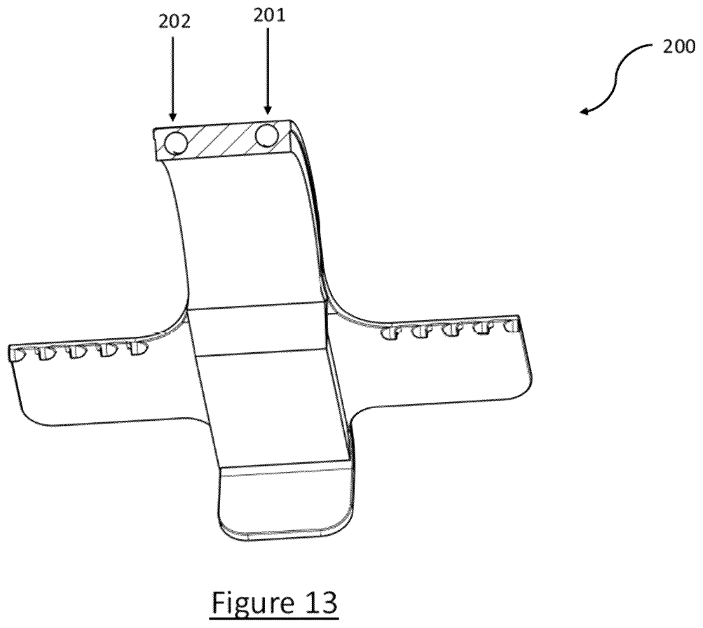

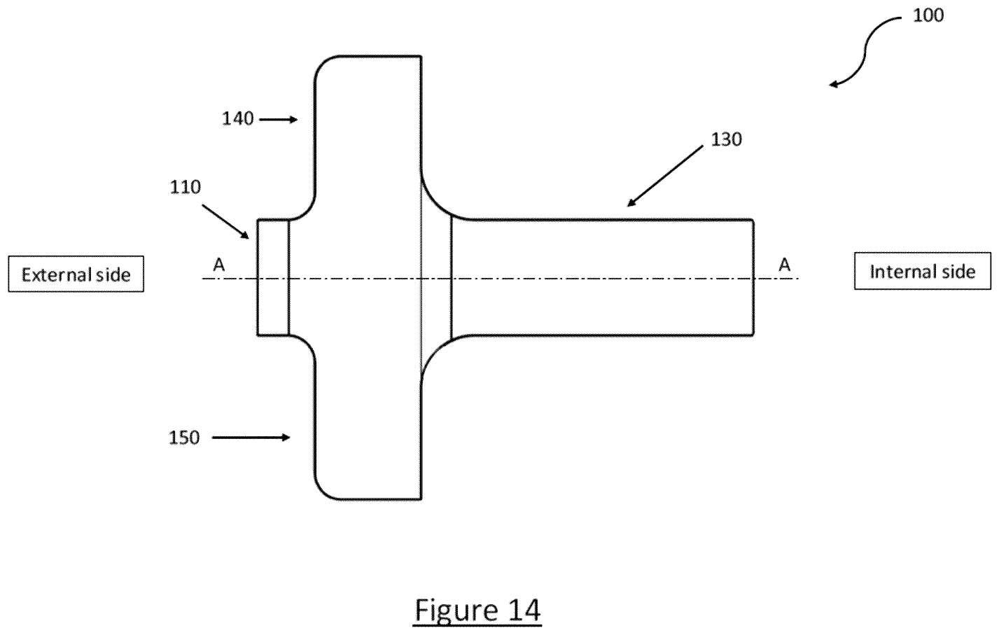

FIGS. 11, 12 & 13 show an embodiment of the oropharyngeal cannula with two oxygen channels 201 & 202, indicated generally by 200. In the present embodiment, two oxygen channels 201 & 202 extend through the interior of the body 120 and the tongue holder 130. The proximal openings 211 & 212 are within the flange 110 and the distal openings 221 & 222 are within the tongue retainer 130. When connected to an oxygen source, the oxygen channels 201 & 202 are configured to deliver oxygen directly into the oropharyngeal space. The number of oxygen channels can vary on different embodiments of the cannula 100.

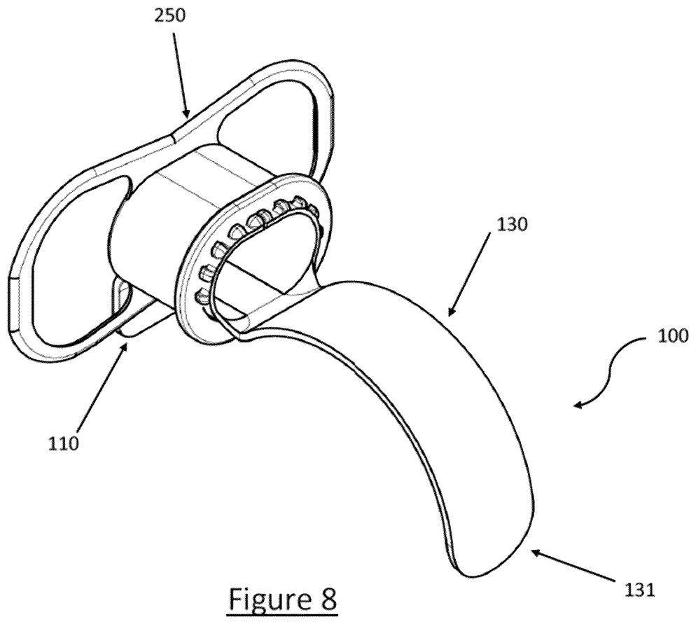

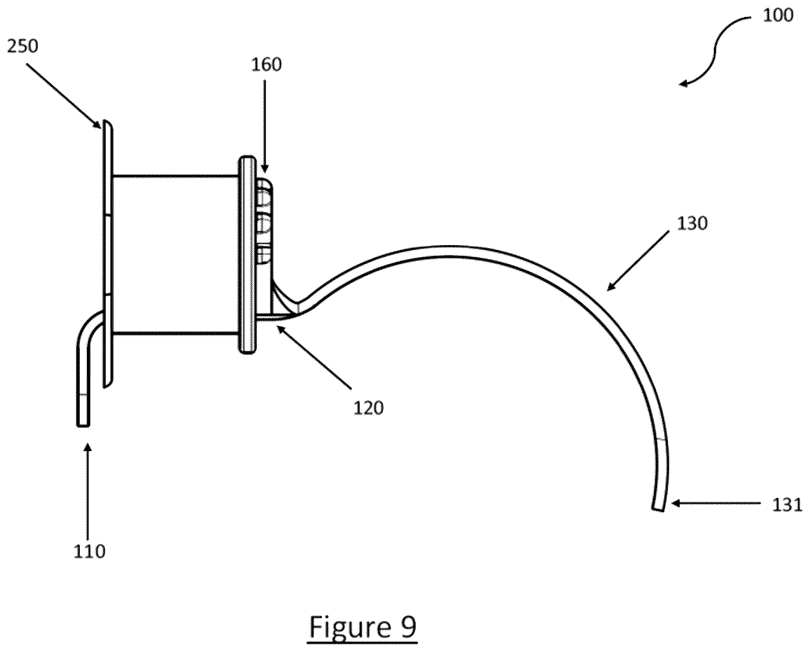

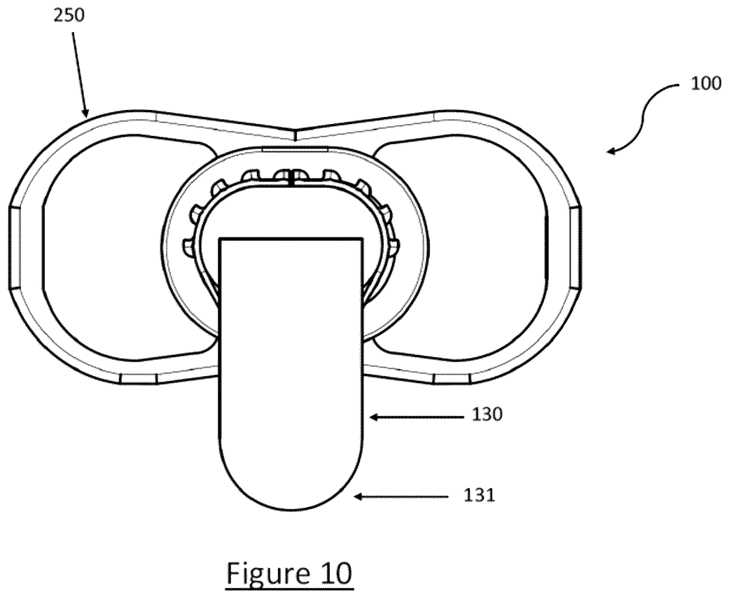

The oropharyngeal cannula insertion process is now described in greater detail. FIGS. 1, 2, 3 & 4 represent the oropharyngeal cannula 100 in its resting configuration. In this configuration, the wings 140 & 150 are flat and rest on the same plane as the body 120. A bite guard 250 (also referred to herein as a bite block) is placed in the patient's mouth before proceeding with anesthesia or sedation. As can be seen in the Figures, the bite guard 250 includes a central channel (also called a bite guard canal) into which the oropharyngeal cannula 100 is designed to be inserted. The oropharyngeal cannula 100 is inserted after eliminating the gag reflex, with anesthesia or sedation, by passing tongue retainer 130 through the bite guard channel and into the patient's mouth. When inserting the oropharyngeal cannula 100, the internal edges 143 & 153 will contact the external surface of the bite guard. As the operator continues applying pressure on the flange 110, the wings 140 & 150 will fold to adapt to the inner shape of the bite guard opening. Thanks to the curved shape of the internal edge 143 & 153 and the convex shape of the sweep hooks 160 & 170, the transition from the unfolded disposition to the folded disposition of the wings 140 & 150 may be smooth and with minimal friction. As the operator continues advancing the cannula 100 by applying pressure on the flange 110, the rim of sweep hooks 160 & 170 will protrude beyond the internal edge of the bite guard 250. This final state of the oropharyngeal cannula is represented by FIGS. 6 & 7. FIGS. 8, 9 & 10 demonstrate the folded oropharyngeal cannula inside a bite guard. In embodiments, and as demonstrated in FIGS. 8, 9 & 10, the oropharyngeal cannula 100 includes: a first portion (e.g., wings 140 & 150) configured to engage the bite guard 250 while positioned in a channel extending through the bite guard 250, wherein the first portion defines a passageway inside the channel; and a second portion (e.g., tongue retainer 130) extending from the first portion and configured to directly contact a superior surface of a tongue of a patient, while the bite guard is in a mouth of the patient, to prevent collapse of an oropharyngeal airway of the patient. In accordance with aspects of the invention, the passageway defined by the first portion (i.e., channel defined by the folded wings 140 & 150 inside the bite guard 250) is sized to accommodate passing an endoscope through the passageway. For example, the oropharyngeal cannula 100 may be sized and shaped such that the passageway defined by the folded wings 140 & 150 is 14 mm across at its smallest dimension such that the passageway can accommodate an endoscope having an outer diameter of 13 mm. In this manner, the oropharyngeal cannula 100 provides a passageway for an endoscope (not shown) to be passed through the channel of the bite guard 250 concurrently with the oropharyngeal cannula 100 being positioned in the channel of the bite guard 250, which provides the benefit of simultaneously maintaining the airway and using a bite guard while performing an endoscopy procedure. The oropharyngeal cannula 100 is not limited to use with the bite guard 250 shown and may be used with different bite guards having differently shaped channels through the bite guard. For example, a bite guard may have a central channel in the shape or a circle, ellipse, or superellipse, and the wings 140 & 150 will curve to the shape of the channel when the oropharyngeal cannula 100 is advanced into the channel in the manner described herein.

If an oropharyngeal cannula with an oxygen channel is being used, as an example of this embodiment is shown at 200 in FIGS. 11, 12 & 13, the cannula 200 can be connected to an oxygen source after the insertion of the cannula 200.

This cannula is intended to adapt to most of the endoscopic bite guards available on the market to this date. Variations of the cannula are contemplated. For example, although in the present embodiment all the sweep hooks 160 & 170 have the same height, it is contemplated that they can vary in height to allow a better adherence with the internal wall of the bite guard. The shape of the sweep hooks 160 & 170 can have other suitable shapes instead of the dome shape defined in this embodiment. These could be such as pyramid shape, cube shape, cone shape or triangle shape, provided that they help oppose the disinsertion forces.

As a further example, although in the present embodiment the tongue holder 130 is defined by a plane surface with a superior convexity, it is contemplated that the plane surface could take on other shapes, such as a concave plane, a half-cylindrical or a closed cylinder, to accommodate the cylindric shape of the endoscope. In such embodiments, the tongue holder 130 may comprise a variety of shapes suitable for accommodating the endoscope, as would be understood by a person skilled in the art.

Thus, it can be seen that the oropharyngeal cannula can be inserted into most of the know bite guards to date after sedation. This oropharyngeal cannula can help prevent the oropharyngeal airway collapse by holding the tongue down and forward. Its novel structure allows the insertion of an endoscopy device while offering an open view of the oropharyngeal tract. When using an oropharyngeal cannula with oxygen tunnels, delivery of oxygen directly into the oropharyngeal space can be ensured.

The above-described embodiments of the invention are intended to be examples of the present invention and alterations and modifications may be affected thereto, by those of the skill in the art, without departing from the scope of the invention.

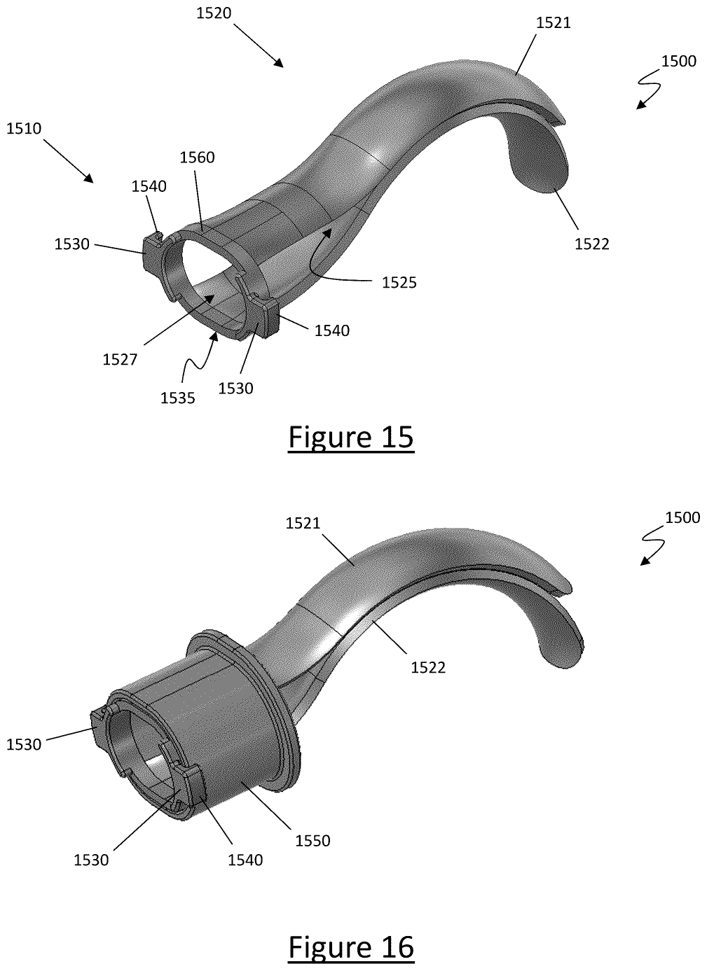

FIGS. 15-22 show additional embodiments of an oropharyngeal cannula designed to be used with a bite guard in accordance with further aspects of the invention. These embodiments are directed to a device that is used to insert into a bite block while the patient is sedated. In various embodiments, the device comprises a front adaptor that is used to fix to the bite block when inserted, an upper guide, and a lower guide. In various embodiments, the device is designed so that it can be inserted into a bite block and turned in multiple directions within the bite block before fixing the front adaptor to the bite block. In accordance with aspects described herein, the upper and lower guides are designed to expand and accommodate an endoscope when the endoscope is inserted through the oropharyngeal cannula while the oropharyngeal cannula is inserted through the bite block. The device thus described maintains the patient airway with or without the endoscope inserted.

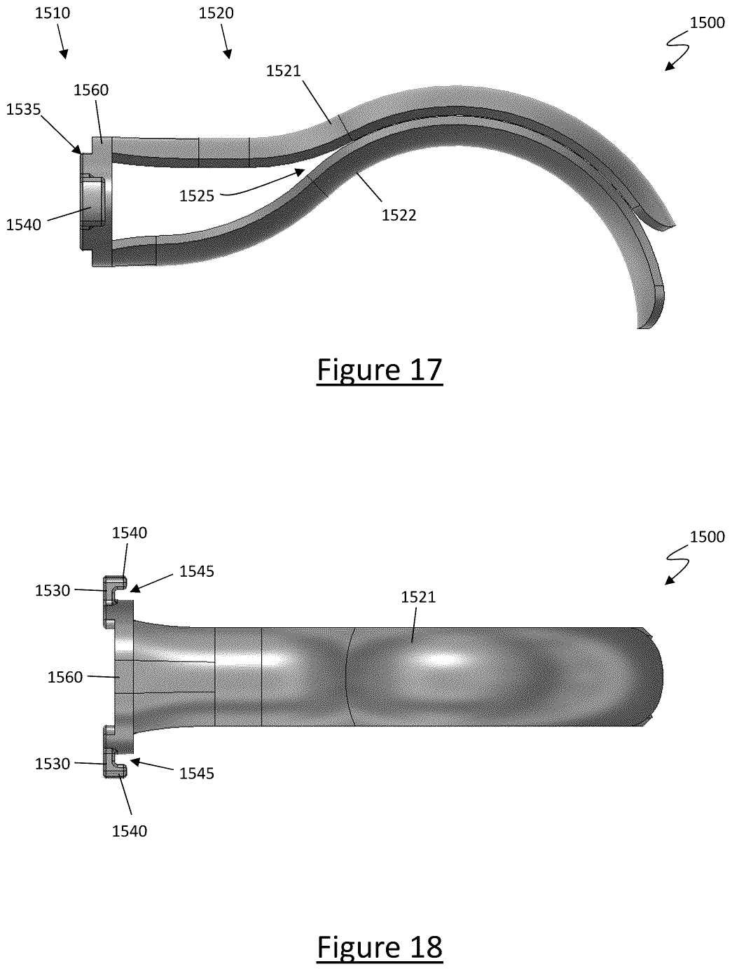

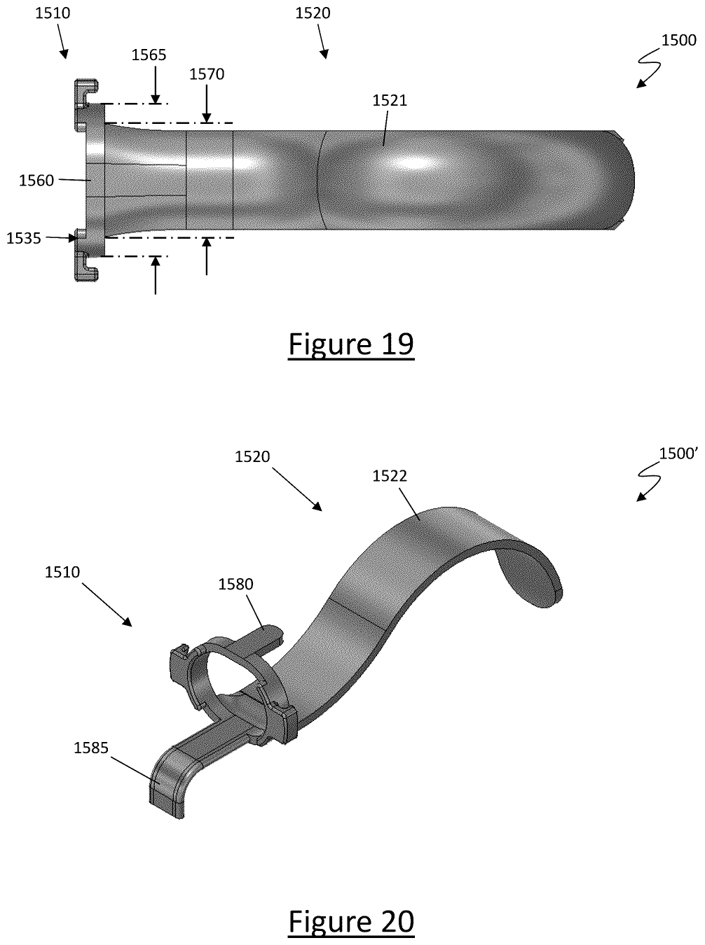

FIGS. 15-19 show an oropharyngeal cannula 1500 configured to be used with a bite guard in accordance with aspects of the invention. FIG. 15 shows the oropharyngeal cannula 1500 without a bite guard, while FIG. 16 shows the oropharyngeal cannula 1500 inserted into and engaged with an exemplary bite guard 1550. FIG. 17 shows a side view of the oropharyngeal cannula 1500. FIGS. 18 and 19 show top-down views of the oropharyngeal cannula 1500. In embodiments, the oropharyngeal cannula 1500 comprises a front adaptor 1510 and a guide 1520, wherein the guide 1520 comprises an upper guide 1521 and a lower guide 1522. The upper guide 1521 and a lower guide 1522 may be sized and shaped to define a guide passageway 1525 between the upper guide 1521 and a lower guide 1522. In various embodiments, the front adaptor 1510 constitutes a first portion of the oropharyngeal cannula 1500 that is configured to engage the bite guard 1550 while positioned in a channel extending through the bite guard 1550, wherein the first portion defines a passageway 1527 inside the channel. In various embodiments, the guide 1520 constitutes a second portion of the oropharyngeal cannula 1500 that extends from the first portion and is configured to directly contact a superior surface of a tongue of a patient, while the bite guard 1550 is in a mouth of the patient, to prevent collapse of an oropharyngeal airway of the patient.

In accordance with aspects of the invention, the passageway 1527 defined by the front adaptor 1510 is sized to accommodate passing an endoscope through the passageway. For example, the oropharyngeal cannula 1500 may be sized and shaped such that the passageway defined by the front adaptor 1510 is 14 mm across at its smallest dimension such that the passageway can accommodate an endoscope having an outer diameter of 13 mm. In this manner, the oropharyngeal cannula 1500 provides a passageway for an endoscope (not shown) to be passed through the channel of the bite guard 1550 concurrently with the oropharyngeal cannula 1500 being positioned in the channel of the bite guard 1550, which provides the benefit of simultaneously maintaining the airway and using a bite guard while performing an endoscopy procedure. The oropharyngeal cannula 1500 is not limited to use with the bite guard 1550 shown and may be used with different bite guards having differently shaped channels through the bite guard.

According to aspects of the invention, the oropharyngeal cannula 1500 may be used with the bite guard in the following manner. First, the bite guard 1550 (also known as a bite block) is placed in the patient's mouth before proceeding with anesthesia or sedation. As can be seen in FIG. 16, the bite guard 1550 includes a central channel (also called a bite guard canal) into which the oropharyngeal cannula 1500 is designed to be inserted. The oropharyngeal cannula 1500 is inserted into the bite guard 1550 after eliminating the gag reflex, with anesthesia or sedation, by passing the guide 1520 through the bite guard channel and into the patient's mouth. In embodiments, the front adaptor 1510 is designed to engage the bite guard 1550 via friction fit (also called interference fit or press fit), and insertion of the oropharyngeal cannula 1500 into the bite guard 1550 continues until the front adaptor 1510 engages the bite guard 1550 by this friction fit. In this configuration, with the bite guard 1550 in the patient's mouth, and with the oropharyngeal cannula 1500 extending through the bite guard 1550 and the front adaptor 1510 being engaged with the bite guard 1550, the guide 1520 of the oropharyngeal cannula 1500 extends into the patient's throat and directly contacts the superior surface of the patient's tongue, including the back one-third of the tongue in the area of the oropharynx, thereby maintaining the patient's airway. In this configuration, an endoscope (or other type of scope) may be passed through the passageway defined by the oropharyngeal cannula 1500 to perform an endoluminal exploration procedure through the mouth of the patient.

In embodiments, the front adaptor 1510 is designed to engage the bite guard 1550 via friction fit. Various structures can be used to create such a friction fit system, and implementations of the oropharyngeal cannula 1500 are not limited to any one particular size and shape of such a structure. FIGS. 15-19 show a non-limiting example of such a structure that includes engagement tabs 1530 that extend laterally from a body 1535 of the front adaptor 1510, with flanges 1540 extending rearwardly from the engagement tabs 1530. As shown in FIGS. 15, 16, and 18, the body 1535, tabs 1530, and flanges 1540 define an engagement space 1545 that is sized and shaped to receive and engage a portion of the bite guard 1550 via friction fit.

In accordance with aspects of the invention, the body 1535 of the front adaptor 1510 comprises an outer surface 1560 having a shape that corresponds to a shape of an interior surface of the channel of the bite guard 1550 and that directly contacts the interior surface of the channel of the bite guard 1550 when the front adaptor 1510 engages the bite guard 1550. The oropharyngeal cannula 1500 is not limited to use with the bite guard 1550 shown and may be designed for use with different bite guards having differently shaped channels through the bite guard. For example, a particular bite guard may have a central channel in the shape or a circle, ellipse, or superellipse, and the body 1535 may be designed and manufactured with a shape and outer surface 1560 corresponding to the central channel of the particular bite guard.

According to aspects of the invention, the front adaptor 1510 has a size and shape that prevents rotation of the oropharyngeal cannula 1500 relative to the bite guard 1550 when the front adaptor 1510 is engaged with the bite guard, and the guide 1520 has a size and shape that permits rotation of the oropharyngeal cannula 1500 relative to the bite guard 1550 when the guide 1520 is inside the channel of the bite guard 1550 and the front adaptor 1510 is not engaged with the bite guard 1550. For example, as shown in FIG. 19, the outer surface 1560 of the body 1535 of the front adaptor 1510 may be sized and shaped with a dimension 1565 that is only slightly smaller than a dimension of an interior surface of the channel of the bite guard 1550, such that the outer surface 1560 contacts the interior surface of the channel of the bite guard 1550 when the front adaptor 1510 is engaged with the bite guard 1550, and wherein this surface-to-surface contact prevents rotation of the oropharyngeal cannula 1500 relative to the bite guard 1550. As additionally shown in FIG. 19, the guide 1520 may be sized and shaped with a dimension 1570 that is sufficiently smaller than the dimension 1565 and that permits rotation of the oropharyngeal cannula 1500 relative to the bite guard 1550 when the front adaptor 1510 is engaged with the bite guard 1550, e.g., when the outer surface 1560 of the body 1535 is not contacting the interior surface of the channel of the bite guard 1550. This rotation permits the user to rotate the oropharyngeal cannula 1500 relative to the bite guard 1550 when inserting the oropharyngeal cannula 1500 into the bite guard 1550, which is helpful for advancing the guide 1520 into the throat of the patient during such insertion.

In various embodiments, and with continued reference to FIGS. 15-17, the upper guide 1521 and the lower guide 1522 define a guide passageway 1525 between the upper guide 1521 and the lower guide 1522. The guide passageway 1525 assists in guiding an endoscope through the guide 1520 during an endoluminal exploration procedure. In some embodiments, at least one of the upper guide 1521 and the lower guide 1522 is resiliently affixed to (or resiliently formed with) the front adaptor 1510. In this manner, one or both of the upper guide 1521 and the lower guide 1522 may flex relative to the front adaptor 1510 when an endoscope having an outer diameter greater than a dimension of the guide passageway 1525 is advanced through the guide passageway 1525. This provides the benefit of the guide 1520 having a relatively smaller initial shape that assists in advancing the guide 1520 into the patient's throat when the oropharyngeal cannula 1500 is inserted into the bite guard 1550, and the guide 1520 being expandable to a relatively larger flexed shape that accommodates an endoscope passing through the guide 1520 when the guide is located in the patient's throat.

FIGS. 20-22 illustrate variations of the oropharyngeal cannula of FIGS. 15-19 according to embodiments of the invention. FIG. 20 shows a variation of the oropharyngeal cannula 1500′ in which the guide 1520 includes the lower guide 1522 but does not include the upper guide 1521. The front adaptor 1510 of the oropharyngeal cannula 1500′ of FIG. 20 also includes a support tab 1580 that is configured to contact the interior surface of the channel of the bite guard 1550 when the front adaptor 1510 is engaged with the bite guard 1550 to provide more surface area of the engagement for the purpose of increasing the stability of the engagement. The oropharyngeal cannula 1500′ of FIG. 20 also includes a flange 1585 extending outward from the front adaptor 1510 on a side of the front adaptor 1510 opposite the guide 1520. The flange 1585 may be used to pull the oropharyngeal cannula 1500′ forward in the patient's mouth, for example, to pull the tongue forward in the throat. The flange 1585 may also be used to apply a force to disengage the oropharyngeal cannula 1500′ from the bite guard 1550, for example, at the conclusion of the procedure. A flange similar to flange 1585 may be included as part of the oropharyngeal cannula 1500 of FIGS. 15-19.

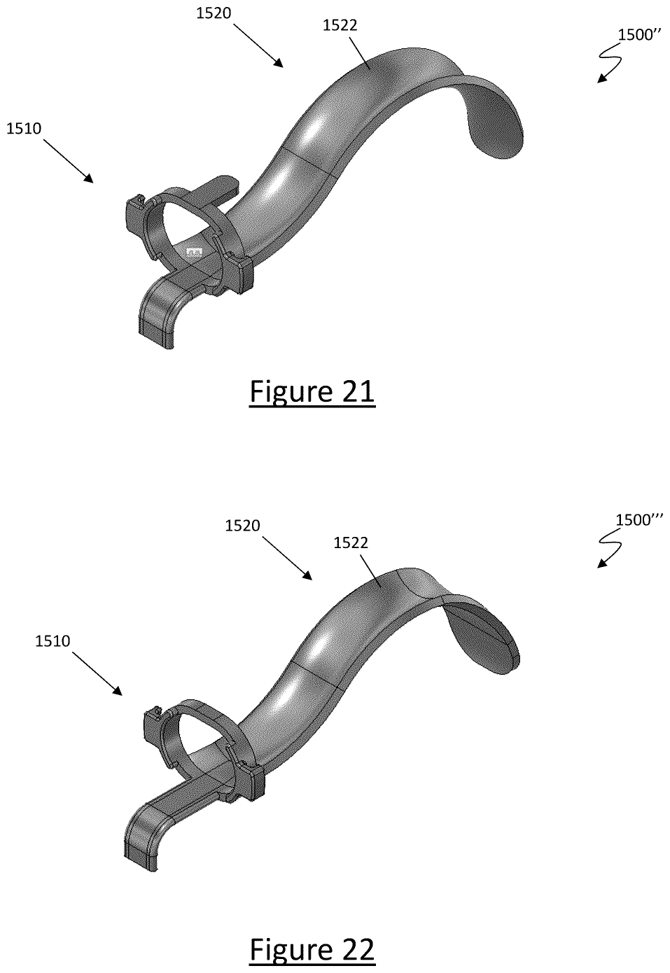

FIGS. 21 and 22 show respective variations of the oropharyngeal cannula similar to the oropharyngeal cannula 1500′ of FIG. 20 but with a differently shaped lower guides 1522. The lower guide 1522 of the oropharyngeal cannula 1500′ of FIG. 20 includes a flat lower surface configured to directly contact the superior surface of the tongue of the patient when the first portion is engaged with the bite guard in the mouth of the patient, and a flat upper surface opposite the lower surface. The lower guide 1522 of the oropharyngeal cannula 1500″ of FIG. 21 includes a convex lower surface and a concave upper surface. The lower guide 1522 of the oropharyngeal cannula 1500′″ of FIG. 22 includes a first portion at a proximal end having a convex lower surface and a concave upper surface, and a second portion at the distal end having a flat lower surface and a flat upper surface. Any of these surface shapes of the lower guide may be used with the lower guide 1522 of the oropharyngeal cannula 1500 of FIGS. 15-19.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations of the present invention are not intended to be limited to the particulars disclosed herein; rather, implementations of the present invention extend to all functionally equivalent structures, methods and uses, such as are within the scope of the appended claims.

What is claimed:

1. An oropharyngeal cannula device configured to be inserted into a bite guard, the device comprising:
a first portion configured to engage the bite guard while positioned in a channel extending through the bite guard, wherein the first portion defines a passageway inside the channel; and
a second portion extending from the first portion and configured to directly contact a superior surface of a tongue of a patient, while the bite guard is in a mouth of the patient, to prevent collapse of an oropharyngeal airway of the patient.

2. The device of claim 1, wherein the passageway is sized to accommodate passing an endoscope through the passageway.

3. The device of claim 1, wherein:
the first portion comprises: a body; a first wing extending from a first lateral side of the body; and a second wing extending from a second lateral side of the body; and
the second portion comprises a tongue holder extending from a backside of the body.

4. The device of claim 3, wherein the first wing and the second wing are foldable between a first configuration in which the first wing and the second wing extend laterally outward from the body and a second configuration in which the first wing and the second wing are curved to a shape corresponding to an interior surface of the channel of the bite guard.

5. The device of claim 3, wherein the first wing and the second wing each include one or more protrusions configured to engage an internal wall of the bite guard.

6. The device of claim 3, further comprising a flange extending from a front side of the body.

7. The device of claim 3, further comprising one or more air channels extending continuously through the body and the tongue holder.

8. The device of claim 1, wherein:
the first portion comprises a front adaptor; and
the second portion comprises a guide.

9. The device of claim 8, wherein the guide comprises an upper guide and a lower guide that define a guide passageway between the upper guide and the lower guide.

10. The device of claim 9, wherein:
at least one of the upper guide and the lower guide is resiliently affixed to the front adaptor; and
the at least one of the upper guide and the lower guide is configured to flex relative to the front adaptor in response to an endoscope having an outer diameter greater than a dimension of the guide passageway passing through the guide passageway.

11. The device of claim 8, wherein the front adaptor is configured to engage the bite guard via friction fit.

12. The device of claim 8, wherein the front adaptor comprises an outer surface having a shape that corresponds to a shape of an interior surface of the channel of the bite guard and that directly contacts the interior surface of the channel of the bite guard when the front adaptor engages the bite guard.

13. The device of claim 8, wherein:
the front adaptor has a size and shape that prevents rotation of the device relative to the bite guard when the front adaptor is engaged with the bite guard; and
the guide has a size and shape that permits rotation of the device relative to the bite guard when the guide is inside the channel of the bite guard and the front adaptor is not engaged with the bite guard.

14. The device of claim 8, wherein the guide comprises:
a lower surface configured to directly contact the superior surface of the tongue of the patient when the first portion is engaged with the bite guard in the mouth of the patient; and
an upper surface opposite the lower surface.

15. The device of claim 14, wherein the lower surface and the upper surface are flat.

16. The device of claim 14, wherein the lower surface is convex and the upper surface is concave.

17. The device of claim 14, wherein:
a first portion of the lower surface is convex and a second portion of the lower surface is flat; and
a first portion of the upper surface is concave and a second portion of the upper surface is flat.

18. The device of claim 8, further comprising a flange extending outward from the front adaptor on a side of the front adaptor opposite the guide.

19. A method of using the oropharyngeal cannula device of claim 1, the method comprising:
inserting the oropharyngeal cannula device into the bite guard that is in the mouth of the patient, wherein the inserting comprises advancing the second portion of the oropharyngeal cannula device through the channel of the bite guard and causing the first portion of the oropharyngeal cannula device to engage the bite guard.

20. The method of claim 19, further comprising passing an endoscope through the passageway of the oropharyngeal cannula device while the oropharyngeal cannula device is inside the channel of the bite guard.
